(19) United States
(12) Patent Application Publication
(10) Pub. No.: US 2023/0287475 A1
(43) Pub. Date: Sep. 14, 2023

(54) DE-CROSSLINKING COMPOUNDS AND METHODS OF USE FOR SPATIAL ANALYSIS

(71) Applicant: 10x Genomics, Inc., Pleasanton, CA (US)

(72) Inventors: James Michael Chell, Stockholm (SE); Joshua Delaney, Northcote (AU); Rapolas Spalinskas, Solna (SE); Aleksandra Jurek, Stockholm (SE); Meiliana Tjandra, Dublin, CA (US)

(21) Appl. No.: 18/018,101

(22) PCT Filed: Dec. 22, 2020
(86) PCT No.: PCT/US2020/066681

§ 371 Date: Jan. 26, 2023

Related U.S. Application Data
(60) Provisional application No. 63/059,535, filed on Jul. 31, 2020.

(51) Int. Cl. C12Q 1/6806

(52) U.S. Cl. CPC C12Q 1/6806

(57) ABSTRACT

Provided herein are methods for de-crosslinking fixed biological samples (e.g., fixed biological samples including aminal crosslinks). The compositions and methods disclosed can de-crosslink oligonucleotides (e.g., DNA or RNA) or proteins from fixed biological samples (e.g., fixed biological samples with aminal crosslinks), wherein the de-crosslinked biological sample is compatible with and can be used in spatial gene expression analysis.

### CROSS-REFERENCE TO RELATED APPLICATIONS

This application claims priority to U.S. Application Ser. No. 63/059,535, filed on Jul. 31, 2020, which is incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Formaldehyde fixation is a common form of tissue preservation, and after formaldehyde fixation, tissues are often embedded in paraffin wax (FFPE) for long term storage. Formaldehyde fixation crosslinks amine functional groups of nucleic acids and proteins through covalent linkage.

### SUMMARY

Crosslinked amine functional groups (e.g., in aminal crosslinks) are typically not compatible with gene expression analysis, including spatial gene expression analysis. Traditionally, heat has been used to try to break the crosslinks of formaldehyde-fixed tissues. This disclosure is based on, at least in part, the identification and use of de-crosslinking reagents that are suitable for use with crosslinked biological samples for spatial analysis. In one aspect, provided herein is a method of producing a de-crosslinked biological sample, the method including (a) contacting a fixed biological sample with a substrate including a plurality of capture probes, wherein a capture probe of the plurality of capture probes includes a capture domain, and (b) contacting the fixed biological sample with a de-crosslinking agent, thereby producing the de-crosslinked biological sample.

In one aspect, provided herein is a method of producing a de-crosslinked biological sample, the method including (a) contacting a fixed biological sample with a substrate including a plurality of capture probes, wherein a capture probe includes a capture domain, and (b) contacting the fixed biological sample with a de-crosslinking agent, thereby producing the de-crosslinked biological sample.

In another aspect, provided herein is a method of producing a de-crosslinked biological sample, the method including (a) contacting a fixed biological sample with a de-crosslinking agent, thereby producing the de-crosslinked biological sample, and (b) contacting the de-crosslinked biological sample with a substrate including a plurality of capture probes, wherein a capture probe of the plurality of capture probes includes a capture domain.

In another aspect, provided herein is a method of producing a de-crosslinked biological sample, the method including (a) contacting a fixed biological sample with a de-crosslinking agent, thereby producing the de-crosslinked biological sample, and (b) contacting the de-crosslinked biological sample with a substrate including a plurality of capture probes, wherein a capture probe includes a capture domain.

In some embodiments, the de-crosslinking agent can be a compound of Formula (I) or a compound of Formula (II). In some embodiments, the de-crosslinking agent can be selected from the group consisting of compounds (1)-(18). In some embodiments, the de-crosslinking agent can be a compound of Formula (I). In some embodiments, the de-crosslinking agent can be selected from the group consisting of compounds (1)-(14). In some embodiments, the de-crosslinking agent can be selected from the group consisting of compounds (1)-(11). In some embodiments, the de-crosslinking agent can be a compound of Formula (II). In some embodiments, the decrosslinking agent is selected from the group consisting of compounds (15)-(18). In some embodiments, the de-crosslinking agent can be compound (1). In some embodiments, the de-crosslinking agent can be contacted to the fixed biological sample at a concentration of about 10 mM to about 500 mM. In some embodiments, the de-crosslinking agent can be contacted to the fixed biological sample at a concentration of about 10 mM to about 100 mM. In some embodiments, the de-crosslinking agent can be contacted to the fixed biological sample at a concentration of about 50 mM to about 150 mM. In some embodiments, the de-crosslinking agent can be contacted to the fixed biological sample at a concentration of about 30 mM to about 70 mM. In some embodiments, the de-crosslinking agent can be contacted to the fixed biological sample at a concentration of about 40 mM to about 60 mM. In some embodiments, the de-crosslinking agent can be contacted to the fixed biological sample at a concentration of about 50 mM.

In some embodiments, the step of contacting the fixed biological sample with the de-crosslinking agent can be performed for about 1 minute to about 120 minutes. In some embodiments, the step of contacting the fixed biological sample with the de-crosslinking agent can be performed for about 30 minutes to about 90 minutes. In some embodiments, the step of contacting the fixed biological sample with the de-crosslinking agent can be performed for about 60 minutes.

In some embodiments, the step of contacting the fixed biological sample with the de-crosslinking agent can be performed at a temperature of about 45° C. and about 95° C. In some embodiments, the step of contacting the fixed biological sample with the de-crosslinking agent can be performed at a temperature of about 60° C. to about 80° C. In some embodiments, the step of contacting the fixed biological sample with the de-crosslinking agent can be performed at a temperature of about 70° C.

In some embodiments, the de-crosslinking agent can be present in a solution or a suspension. In some embodiments, the solution or suspension further includes a buffer. In some embodiments, the buffer can be selected from the group consisting of: tris(hydroxymethyl)aminomethane (Tris), tris(hydroxymethyl)aminomethane-Ethylenediaminetetraacetic acid (TE), phosphate-buffered saline (PBS), 2-[4-(2-hydroxyethyl)piperazin-1-yl]ethanesulfonic acid (HEPES), 2-morpholin-4-ylethanesulfonic acid (MES), and a combination thereof. In some embodiments, the buffer can be present in the solution or the suspension at a concentration of about 5 mM to about 50 mM. In some embodiments, the buffer can be present in the solution or the suspension at a concentration of about 20 mM to about 40 mM. In some embodiments, the buffer can be present in the solution or the suspension at a concentration of about 30 mM.

In some embodiments, the fixed biological sample can be a paraffinized fixed biological sample. In some embodiments, method further includes, prior to the step of contacting the paraffinized fixed biological sample with the de-crosslinking agent, a step of deparaffinizing the paraffinized fixed biological sample, thereby producing a de-paraffinized fixed biological sample, and optionally, rehydrating the de-paraffinized fixed biological sample. In some embodiments, the step of deparaffinizing the paraffinized fixed biological sample includes contacting the paraffinized fixed biological sample with xylene and ethanol. In some embodiments, the step of rehydrating the de-paraffinized fixed biological sample includes contacting the de-paraffinized fixed biological sample with water. In some embodiments, the step of deparaffinizing the paraffinized fixed biological sample includes, contacting the paraffinized fixed biological sample with xylene, absolute ethanol, about 96% ethanol, and about 70% ethanol. In some embodiments, the step of deparaffinizing the paraffinized fixed biological sample includes, sequentially, contacting the paraffinized fixed biological sample with xylene, absolute ethanol, about 96% ethanol, and about 70% ethanol.

In some embodiments, the method further includes, prior to the step of contacting the fixed biological sample with the de-crosslinking agent, a step of pretreating the fixed biological sample. In some embodiments, the step of pretreating the fixed biological sample includes contacting the fixed biological sample with a proteinase. In some embodiments, the proteinase can be present in a solution or a suspension. In some embodiments, the proteinase can be a collagenase. In some embodiments, the proteinase can be present in the solution or the suspension at a concentration of about 0.05 to about 0.5 U/μL. In some embodiments, the proteinase can be present in the solution or the suspension at a concentration of about 0.1 to about 0.3 U/μL. In some embodiments, the proteinase can be present in the solution or the suspension at a concentration of about 0.2 U/μL. In some embodiments, the solution or the suspension further includes a detergent. In some embodiments, the detergent can be a non-ionic detergent. In some embodiments, the detergent can be 2-[4-(2,4,4-trimethylpentan-2-yl)phenoxy]ethanol. In some embodiments, the detergent can be present in the solution or the suspension at a concentration of about 0.05% (v/v) to about 2% (v/v). In some embodiments, the detergent can be present in the solution or the suspension at a concentration of about 0.1% (v/v) to about 1% (v/v). In some embodiments, the detergent can be present in the solution or the suspension at a concentration of about 0.5% (v/v). In some embodiments, the solution or suspension further includes a buffer. In some embodiments, the buffer can be Hank's Balanced Salt Solution (HBSS) buffer. In some embodiments, the buffer can be TE buffer. In some embodiments, the buffer includes Tris, TE, PBS, HEPES, MES, or a combination thereof. In some embodiments, the solution or the suspension has a pH of about 7.0 to about 9.0. In some embodiments, the solution or the suspension has a pH of about 7.5 to about 8.5. In some embodiments, the solution or the suspension has a pH of about 8.0.

In some embodiments, the method further includes, after the step of contacting the fixed biological sample with the de-crosslinking agent, a step of permeabilizing the de-crosslinked biological sample.

In some embodiments, the fixed biological sample can be an aldehyde-fixed biological sample. In some embodiments, the fixed biological sample can be a formaldehyde-fixed biological sample. In some embodiments, the fixed biological sample can be a formaldehyde-fixed paraffin-embedded (FFPE) biological sample. In some embodiments, the fixed biological sample can be a fixed tissue section.

In some embodiments, the method further includes, prior to the step of contacting the fixed biological sample with the de-crosslinking agent, a step of staining the fixed biological sample, and imaging the fixed biological sample. In some embodiments, the method further includes, after the step of contacting the fixed biological sample with the de-crosslinking agent, a step of staining the de-crosslinked biological sample, and imaging the de-crosslinked biological sample. In some embodiments, the staining includes the use of a histological stain. In some embodiments, the staining includes the use of an immunological stain.

In some embodiments, the substrate includes a slide. In some embodiments, the substrate includes a bead.

In some embodiments, the capture probe further includes a unique molecular identifier (UMI). In some embodiments, the capture probe includes DNA. In some embodiments, the capture domain includes a poly(T) sequence. In some embodiments, the capture probe further includes a spatial barcode.

In another aspect, provided herein is a method of determining a location of a nucleic acid analyte in the fixed biological sample, the method including (i) producing a de-crosslinked biological sample according to any of the methods described herein, wherein the capture domain binds specifically to the nucleic acid analyte of the biological sample, and (ii) determining (I) a sequence corresponding to the nucleic acid analyte or a complement thereof, and (II) a sequence corresponding to the spatial barcode of the capture probe or a complement thereof, and using the determined sequences of (I) and (II) to determine the location of the nucleic acid analyte in the fixed biological sample.

In another aspect, provided herein is a method of determining a location of a nucleic acid analyte in the fixed biological sample, the method including (i) producing a de-crosslinked biological sample according to any of the methods described herein, wherein the capture domain binds to the nucleic acid analyte of the biological sample, and (ii) determining (I) a sequence corresponding to the nucleic acid analyte or a complement thereof, and (II) a sequence corresponding to the spatial barcode of the capture probe or a complement thereof, and using the determined sequences of (I) and (II) to determine the location of the nucleic acid analyte in the fixed biological sample.

In some embodiments, the nucleic acid analyte can be DNA. In some embodiments, the nucleic acid analyte can be an RNA. In some embodiments, the RNA can be an mRNA. In some embodiments, the method further includes extending an end of the capture probe using the nucleic acid analyte as a template.

In another aspect, provided herein is a method of determining the location of a protein analyte in the fixed biological sample, the method including (i) contacting a de-crosslinked biological sample on a substrate including a plurality of capture probes, wherein a capture probe of the plurality of capture probes includes a capture domain and a spatial barcode, with a plurality of analyte capture agents, wherein an analyte capture agent of the plurality of analyte capture agents includes (1) an analyte binding moiety that binds specifically to the protein analyte from the fixed biological sample, (2) an analyte binding moiety barcode, and (3) an analyte capture sequence, wherein the analyte capture sequence binds specifically to the capture domain of the capture probe, and (ii) determining (I) a sequence corresponding to the analyte binding moiety barcode or a complement thereof, and (II) a sequence corresponding to the spatial barcode of the capture probe or a complement thereof, and using the determined sequences of (I) and (II) to determine the location of the protein analyte in the fixed biological sample.

In another aspect, provided herein is a method of determining the location of a protein analyte in the fixed biological sample, the method including (i) contacting a de-crosslinked biological sample on a substrate including a plurality of capture probes, wherein a capture probe includes a capture domain and a spatial barcode, with a plurality of analyte capture agents, wherein an analyte capture agent includes (1) an analyte binding moiety that binds specifically to the protein analyte from the fixed biological sample, (2) an analyte binding moiety barcode, and (3) an analyte capture sequence, wherein the analyte capture sequence binds to the capture domain of the capture probe, and (ii) determining (I) a sequence corresponding to the analyte binding moiety barcode or a complement thereof, and (II) a sequence corresponding to the spatial barcode of the capture probe or a complement thereof, and using the determined sequences of (I) and (II) to determine the location of the protein analyte in the fixed biological sample.

In some embodiments, the biological sample can be de-crosslinked using a de-crosslinking agent that is a compound of Formula (I) or a compound of Formula (II). In some embodiments, the biological sample can be de-crosslinked using a de-crosslinking agent selected from the group consisting of compounds (1)-(18). In some embodiments, the biological sample can be de-crosslinked using a de-crosslinking agent that is a compound of Formula (I). In some embodiments, the biological sample can be de-crosslinked using a de-crosslinking agent selected from the group consisting of compounds (1)-(14). In some embodiments, the biological sample can be de-crosslinked using a de-crosslinking agent selected from the group consisting of compounds (1)-(11). In some embodiments, the biological sample can be de-crosslinked using a de-crosslinking agent that is a compound of Formula (II). In some embodiments, the biological sample can be de-crosslinked using a de-crosslinking agent selected from the group consisting of compounds (15)-(18). In some embodiments, the biological sample can be de-crosslinked using compound (1).

In some embodiments, the method includes, prior to (i), producing the de-crosslinked biological sample according any of the methods described herein.

In some embodiments, the protein analyte can be an intracellular protein. In some embodiments, the protein analyte can be an extracellular protein. In some embodiments, the protein analyte can be a cell surface protein.

In some embodiments, the analyte-binding moiety includes an antibody or an antigen-binding domain thereof.

In some embodiments, method further includes extending an end of the capture probe using the analyte binding moiety barcode as a template.

In some embodiments, the determining of the sequences of (I) and (II) includes sequencing of the sequences of (I) and (II). In some embodiments, the sequencing can be high throughput sequencing.

In another aspect, provided herein is a method of producing a de-crosslinked biological sample, the method including (a) contacting a formaldehyde-fixed paraffin-embedded (FFPE) biological sample with a substrate including a plurality of capture probes, wherein a capture probe of the plurality of capture probes includes a capture domain (b) deparaffinizing the FFPE biological sample, (c) staining the FFPE biological sample with hematoxylin and eosin, (d) pre-treating the FFPE biological sample with collagenase, a detergent, or collagenase or a detergent, and (e) contacting the FFPE biological sample with compound (1), thereby producing the de-crosslinked biological sample.

In another aspect, provided herein is a method of producing a de-crosslinked biological sample, the method including (a) contacting a formaldehyde-fixed paraffin-embedded (FFPE) biological sample with a substrate including a plurality of capture probes, wherein a capture probe includes a capture domain (b) deparaffinizing the FFPE biological sample, (c) staining the FFPE biological sample with hematoxylin and eosin, (d) pre-treating the FFPE biological sample with collagenase, a detergent, or collagenase or a detergent, and (e) contacting the FFPE biological sample with compound (1), thereby producing the de-crosslinked biological sample.

In some embodiments, deparaffinizing the FFPE biological sample includes, sequentially, contacting the FFPE biological sample with xylene, absolute ethanol, about 96% ethanol, and about 70% ethanol.

In some embodiments, the FFPE sample can be pre-treated with collagenase at 0.2 U/μL in HBSS buffer for 20 minutes at 37° C.

In some embodiments, the FFPE sample can be pre-treated with 0.5% of a non-ionic detergent in TE buffer at pH 8 for 20 minutes at 37° C.

In some embodiments, the FFPE sample can be contacted with 50 mM compound (1) in Tris or TE buffer for 1 hour at 70° C.

Also provided herein is a kit for practicing any of the methods described herein, the kit including (a) a substrate including a plurality of capture probes, wherein a capture probe compises a spatial barcode and a capture domain, and (b) a reagent including one or more of compounds (1)-(18). In some embodiments, the compound can be compound (1). In some embodiments, the kit can further include (a) one or more polymerize enzymes, (b) one or more wash buffers, and (c) one or more reaction buffers.

Also provided herein are compositions that comprise or consist of compound (1), compound (2), compound (3), compound (4), compound (5), compound (6), compound (7), compound (8), compound (9), compound (10), compound (11), compound (12), compound (13), compound (14), compound (15), compound (16), compound (17), or compound (18).

Also provided herein are compositions that comprise or consist of one or more of: compound (1), compound (2), compound (3), compound (4), compound (5), compound (6), compound (7), compound (8), compound (9), compound (10), compound (11), compound (12), compound (13), compound (14), compound (15), compound (16), compound (17), and compound (18).

As used herein, the term “halo” refers to fluoro (F), chloro (Cl), bromo (Br), or iodo (I).

The term “alkyl” refers to a hydrocarbon chain that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “alkylene” refers to a divalent alkyl (e.g., —CH₂—).

The term “alkenyl” refers to a hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it.

The term “alkynyl” refers to a hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “heteroaryl”, as used herein, means a mono-, bi-, tri- or polycyclic group having 5 to 20 ring atoms, alternatively 5, 6, 9, 10, or 14 ring atoms; and having 6, 10, or 14 pi electrons shared in a cyclic array; wherein at least one ring in the system is aromatic, and at least one ring in the system contains one or more heteroatoms independently selected from the group consisting of N, O, and S (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, furyl, oxazolyl, oxadiazolyl, pyrrolyl, imidazolyl, triazolyl, thiodiazolyl, pyrazolyl, isoxazolyl, thiadiazolyl, pyranyl, pyridyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an exemplary de-crosslinking workflow.

FIG. 2 is a bar plot showing spatial gene expression analysis of formalin-fixed, paraffin-embedded (FFPE) mouse spleen using two different de-crosslinking workflows.

FIG. 3 shows spatial gene expression analysis of formalin-fixed, paraffin-embedded (FFPE) mouse spleen as analyzed by t-SNE clustering. The rendered analysis picture and t-SNE are representations of a real life picture of a mouse spleen gene expression profile and the associated t-SNE analysis. In the left panel, a frame of fiducial markers surrounds the mouse spleen sample.

FIG. 4 is a bar plot showing RNA recovery from de-crosslinking of PBMCs under control and under test conditions including compounds (1)-(6).

FIG. 5 is a bar plot showing RNA recovery from de-crosslinking of overfixed Jurkat cells under control and under test conditions including compounds (3), (8), (12), (13), (14), or (15).

FIG. 6 is a bar plot showing RNA recovery from de-crosslinking of underfixed Jurkat cells under control and under test conditions including compounds (3), (8), (12), (13), (14), or (15).

FIG. 7 is a bar plot showing RNA recovery from de-crosslinking of fixed Jurkat cells under control and under test conditions including compounds (8), (15), (16), (17), or (18).

### DETAILED DESCRIPTION

Unfixed biological samples are typically unstable. When a biological sample is removed from its viable niche, physical decomposition generally begins immediately. The degree of decomposition can be influenced by a number of factors including time, solution buffering conditions, temperature, source (e.g., certain tissues and cells a have higher levels of endogenous RNase activity), biological stress (e.g., enzymatic tissue dissociation can activate stress response genes), and physical manipulation (e.g., pipetting, centrifuging). The degradation can impact nucleic acid molecules (e.g., RNA), proteins, as well as higher-order 3D structure of molecular complexes, whole cells, tissues, organs, and organisms. The instability of biological samples can be a significant obstacle for their use with various analytical methods. Sample degradation can limit the ability to use such methods accurately and reproducibly with a wide range of available biological samples.

In some cases, biological sample instability can be mitigated by preserving or fixing the sample using standard biological preservation methods such as cryopreservation, dehydration (e.g., in methanol), high-salt storage (e.g., using RNAssist or RNAlater), and/or chemical fixing agents that create covalent crosslinks (e.g., paraformaldehyde or DSP). These techniques for stabilizing biological samples can be used alone or in combination. Some methods of preservation can be reversed (e.g., rehydrating a dehydrated sample) more easily than others.

A widely used fixative reagent, paraformaldehyde (PFA), fixes tissue samples by catalyzing crosslink formation (e.g., aminal crosslink formation) between amine groups in analytes, such as the exocyclic amines of adenine, cytosine, or guanosine, or the sidechains of lysine, glutamine, or asparagine in proteins. Both intra-molecular and inter-molecular crosslinks (e.g., aminal crosslinks) can form in an analyte. In some cases, crosslinks (e.g., aminal crosslinks) can preserve protein secondary structure and also eliminate enzymatic activity in the preserved tissue sample. Crosslinking fixatives can be helpful in preserving transient or fine cytoskeletal structure against degradation. The formation of crosslinks (e.g., aminal crosslinks) in analytes (e.g., proteins, RNA, DNA) due to fixation greatly reduces the ability to detect (e.g., bind to, amplify, sequence, hybridize to) these analytes in many standard assay methods. Techniques to remove the crosslinks induced by fixative reagents (e.g., heat, acid) can cause further damage to the analytes (e.g., loss of bases, chain hydrolysis, cleavage, denaturation, etc.). Further description of the consequences of fixation of tissue samples and the benefits of removing adducts and/or crosslinks are described in U.S. Pat. No. 8,288,122, which is hereby incorporated by reference in its entirety.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligation products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein. In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

The ability to use a fixed biological sample in an analytical method, such as a spatial analysis method, is enhanced if the cross-links established during fixation of the biological sample are reversed so that an assay can be carried out before sample degradation occurs. Ideally, data obtained from a de-crosslinked biological sample should be similar to that obtained from a fresh sample.

As used herein, a fixed biological sample can be any appropriate fixed biological sample. See, for example, Section (I)(d) of WO 2020/176788 and/or U.S. Patent Application Publication No. 2020/0277663. In some embodiments, a fixed biological sample can be a fixed tissue sample (e.g., a fixed tissue section). In some embodiments, a fixed biological sample can include aminal crosslinks.

Aminal crosslinks can be made, for example, by fixing a sample with formaldehyde. In some embodiments, the fixative or fixation agent is formaldehyde. The term “formaldehyde” when used in the context of a fixative also refers to “paraformaldehyde” (or “PFA”) and “formalin”, both of which are terms with specific meanings related to the formaldehyde composition (e.g., formalin is a mixture of formaldehyde and methanol). Thus, a formaldehyde-fixed biological sample may also be referred to as formalin-fixed or PFA-fixed. Protocols and methods for the use of formaldehyde as a fixation reagent to prepare fixed biological samples are known in the art, and can be used in the methods and compositions of the present disclosure. In some embodiments, a biological sample is a formalin-fixed, paraffin-embedded (FFPE) tissue sample (e.g., an FFPE tissue section).

In some embodiments, provided herein are methods of de-crosslinking aminal crosslinks in a fixed biological sample. In some embodiments, provided herein are methods of spatial analysis using such a de-crosslinked sample.

In some embodiments, aldehyde fixation methods can be combined with other tissue preservation methods. See, e.g., Section (1)(d)(ii)(1)-(5) of WO 2020/176788 and/or U.S. Patent Application Publication No. 2020/0277663. For example, aldehyde fixation can be combined with fresh frozen preservation of tissues. Aldehyde fixation can be combined with alcohol fixation, or with any number of commercially available fixation/preservation techniques. For example, aldehyde fixation can be combined with salt-rich buffer solutions such as RNAlater, low-temperature preservation buffers such as HypoThermosol, alcohol-PEG fixation (e.g., Neo-Rix, STATFIX, PAGA, UMFIX), PAXGene, Allprotect/Xprotect, CellCover, RNAssist, or zinc buffers.

Fixation (e.g., aldehyde fixation) of biological samples may require adjusting other parameters or workflows described herein. For example, aldehyde fixation used with spatial analysis workflows may require longer permeabilization periods, additional permeabilization reagents, or higher permeabilization reagent concentrations in order to liberate biological analytes (e.g., mRNA) from a cross-linked biological sample for use in spatial analysis workflows described herein. The methods described herein are not limited to any particular fixation reagent that results in crosslinks (e.g., aminal crosslinks) and are equally amenable with any fixation method that results in intra-tissue crosslinking events (e.g., aminal intra-tissue crosslinking events).

(a) Preparation of Fixed Samples for De-Crosslinking

In some embodiments, a biological sample can be provided in a fixed state. In some embodiments, a fixed biological sample can undergo one or more preparation steps before it is pretreated and/or de-crosslinked. See, e.g., Section (1)(d)(ii)(6)-(15) of WO 2020/176788 and/or U.S. Patent Application Publication No. 2020/0277663

For example, in some embodiments, a fixed biological sample (e.g., tissue section) can be incubated on a water bath (e.g., at about 20° C. to about 60° C., about 30° C. to about 50° C., or about 40° C.). In some such embodiments, the fixed biological sample (e.g., tissue section) is subsequently dried (e.g., at about 20° C. to about 60° C., about 30° C. to about 50° C., or about 40° C.) for a period of time (e.g., about 30 minutes to about 4 hours, about 1 hour to about 3 hours, or about 2 hours).

As another example, for paraffin-embedded biological samples (e.g., FFPE samples), the sample can be deparaffinized (e.g., to produce a de-paraffinized fixed biological sample) and rehydrated. In some embodiments, deparaffinizing can include treating with xylene and ethanol (e.g., absolute ethanol, about 96% ethanol, and or about 70% ethanol). In some embodiments, deparaffinization can include, sequentially, treating with xylene (e.g., twice for 7 minutes), treating with absolute ethanol (e.g., twice for two minutes), treating with about 96% ethanol (e.g., once for 2 minutes), and treating with about 70% ethanol (e.g., once for 2 minutes). In some embodiments, this can be followed by treating with water (e.g., twice for 1 minute). See, e.g., Section (1)(d)(ii)(3) of WO 2020/176788 and/or U.S. Patent Application Publication No. 2020/0277663.

(b) Pre-Treating

In some embodiments, a fixed biological sample is pretreated with one or more pretreating reagents prior to delivery or application of a de-crosslinking agent. Pretreatment can include permeabilization of the biological sample, for example, using conditions milder than those typically used for extracting analytes.

In some embodiments, a pretreating reagent can include a proteinase (e.g., collagenase). The proteinase can be present in any appropriate concentration (e.g., about 0.005 to about 0.5 U/μL (e.g., about 0.01 to about 0.5 U/μL, about 0.05 to about 0.5 U/μL, about 0.1 to about 0.5 U/μL, about 0.1 to about 0.3 U/μL, or about 0.2 U/μL). A proteinase can be any appropriate proteinase. In some embodiments, a proteinase can be pepsin, Proteinase K, or an ArcticZymes Proteinase. The proteinase can optionally be applied with a buffer, such as Hank's Balanced Salt Solution (HBSS) buffer. In some embodiments, if pepsin is used for permeabilization, a pretreating reagent can include a proteinase (e.g., a second proteinase or a proteinase other than pepsin). In some embodiments, if Proteinase K is used for permeabilization, a pretreating reagent may not include a proteinase.

In some embodiments, a pretreating reagent can include a detergent. The detergent can be present in any appropriate concentration (e.g., about 0.05% to about 2% (v/v), about 0.1% to about 1% (v/v), about 0.1% (v/v), or about 0.5% (v/v)). In some embodiments, the detergent is a non-ionic detergent. In some embodiments, detergent comprises TRITON™ X100. In some embodiments, the detergent is in a buffer. In some embodiments, the buffer is, for example, tris(hydroxymethyl)aminomethane-Ethylenediaminetetraacetic acid (TE), phosphate-buffered saline (PBS), 2-[4-(2-hydroxyethyl)piperazin-1-yl]ethanesulfonic acid (HEPES), or 2-morpholin-4-ylethanesulfonic acid (MES), with a pH of about 7.0 to about 9.0 (e.g., about 7.5 to about 8.5, or about 8.0).

A pretreating reagent can be applied to the biological sample (e.g., tissue section) in any number of ways. In some embodiments, a pretreating reagent is in solution or suspension. In some embodiments, the biological sample (e.g., tissue section) can be soaked in a solution or suspension comprising a pretreating reagent. In some embodiments, a pretreating reagent is sprayed onto the biological sample (e.g., as a solution or suspension). In some embodiments, a pretreating reagent is supplied to the biological sample (e.g., tissue section) via a microfluidic system (e.g., as a solution or suspension). In some embodiments, the biological sample (e.g., tissue section) is dipped into a solution or suspension of a pretreating reagent, wherein excess solution or suspension is removed from the biological sample (e.g., tissue section). In some embodiments, a pretreating reagent is delivered to the biological sample (e.g., tissue section) via a hydrogel, wherein the hydrogel is a repository for a pretreatment reagent and is contacted with the biological sample. Application of a pretreatment reagent can occur in other ways known in the art.

The pretreatment can be applied to the biological sample for a time sufficient to permeabilize a biological sample so that the de-crosslinking agent can penetrate the biological sample. In some embodiments, the pretreatment can be applied to the biological sample for between about 1 minute and about 60 minutes. In some embodiments, the pretreatment can be applied to the biological sample between about 1 minute and about 55 minutes, about 1 minute and about 50 minutes, about 1 minute and about 45 minutes, about 1 minute and about 40 minutes, about 1 minute and about 35 minutes, about 1 minute and about 30 minutes, about 1 minute and about 25 minutes, about 1 minute and about 20 minutes, about 5 minutes and about 60 minutes, about 10 minutes and about 60 minutes, about 10 minutes and about 50 minutes, about 10 minutes and about 40 minutes, or about 10 minutes and about 30 minutes. In some embodiments, the pretreatment can be applied to the biological sample for about 20 minutes.

The biological sample can be incubated during pretreatment. In some embodiments, the biological sample can be incubated between about 30° C. and about 45° C. In some embodiments, the biological sample can be at about 31° C., 32° C., 33° C., 34° C., 35° C., 36° C., 37° C., 38° C., 39° C., 40° C., 41° C., 42° C., 43° C., 44° C., or 45° C. In some embodiments, the biological sample can be incubated at about 37° C. during pretreatment.

(c) De-Crosslinking Agents

Some conditions for reversing the effects of fixing a biological sample are known in the art, however, these conditions tend to be harsh. See e.g., WO2001/46402; US2005/0014203A1, US2009/0202998A1, Masuda, Norikazu, et al. “Analysis of chemical modification of RNA from formalin-fixed samples and optimization of molecular biology applications for such samples.” Nucleic acids research 27.22 (1999): 4436-4443, Evers, David L., et al. “The effect of formaldehyde fixation on RNA: optimization of formaldehyde adduct removal.” The Journal of Molecular Diagnostics 13.3 (2011): 282-288, and Beechem, Joseph M. “High-Plex spatially resolved RNA and protein detection using digital spatial profiling: A technology designed for immuno-oncology biomarker discovery and translational research.” Biomarkers for Immunotherapy of Cancer. Humana, New York, N.Y., 563-583, each of which is incorporated by reference herein in its entirety. For example, treatment of PFA-treated tissue samples includes heating to 60° C. to 70° C. in Tris buffer for several hours, and yet typically this removes only a fraction of the fixative-induced crosslinks. Furthermore, the harsh de-crosslinking treatment conditions can result in permanent damage to biomolecules (e.g., nucleic acid analytes for analytical methods, such as those described herein) in the sample. Recently, less harsh de-crosslinking techniques and conditions have been proposed that utilize compounds capable of chemically reversing the crosslinks resulting from fixation. See e.g., Karmakar et al., “Organocatalytic removal of formaldehyde adducts from RNA and DNA bases,” Nature Chemistry, 7: 752-758 (2015); US 2017/0283860A1; and US 2019/0135774A1, each of which is incorporated by reference herein in its entirety.

The terms “de-crosslinking agent” (sometimes also called an “un-fixing agent”) as used herein can refer to a compound or composition that reverses fixation and/or removes the crosslinks within or between biomolecules (e.g., analytes for analytical methods, such as those described herein) in a sample caused by previous use of a fixation reagent. In some embodiments, de-crosslinking agents are compounds that act catalytically in removing crosslinks in a fixed sample. In some embodiments, de-crosslinking agents are compounds that act catalytically in removing aminal crosslinks in a fixed sample. In some embodiments, de-crosslinking agents can act on biological samples fixed with an aldehyde (e.g., formaldehyde), an N-hydroxysuccinimide (NHS) ester, an imidoester, or a combination thereof.

In some embodiments, the de-crosslinking agent is a compound of Formula (I):

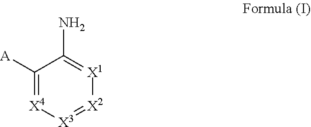

or a pharmaceutically acceptable salt thereof, wherein:
A is selected from the group consisting of: C(═O)OH, P(═O)(OH)₂, and S(═O)₂OH;
X¹, X², X³, and X⁴ are each independently selected from the group consisting of: CH, CR^{a}, and N;
each occurrence of R^{a} is independently selected from the group consisting of: C₁₋₆ alkyl, C₁₋₆ haloalkyl, NO₂, NR′R″, and C(═O)NR′R″; and
each occurrence of R′ and R″ is independently selected from the group consisting of: H and C₁₋₆ alkyl which is optionally substituted with

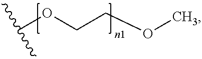

wherein n1 is an integer from 12 to 16.

In some embodiments of Formula (I), it is provided that when A is P(═O)(OH)₂; and X¹, X², and X⁴ are CH, then X³ is other than C—CH₃.

In some embodiments of Formula (I), A is C(═O)OH. In some embodiments of Formula (I), A is P(═O)(OH)₂. In some embodiments of Formula (I), A is S(═O)₂OH.

In some embodiments of Formula (I), X¹ is CH. In some embodiments of Formula (I), X¹ is CR^{a}. In certain of these embodiments X¹ is C—CH₃. In some embodiments of Formula (I), X² is CH. In some embodiments of Formula (I), X² is N. In some embodiments of Formula (I), X⁴ is CH. In some embodiments of Formula (I), X⁴ is N.

In some embodiments of Formula (I), X³ is N. In some embodiments of Formula (I), X³ is CH. In some embodiments of Formula (I), X³ is CR^{a}. In certain of these embodiments, R^{a} is C₁₋₆ alkyl (e.g., methyl). In certain embodiments, R^{a} is NO₂. In certain embodiments, R^{a} is NR′R″ (e.g., NH₂). In certain embodiments, R^{a} is C(═O)NR′R″. As a non-limiting example of the foregoing embodiments, R^{a} is

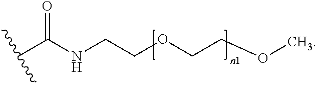

In some embodiments of Formula (I), X² and X⁴ are CH. In some embodiments of Formula (I), X¹, X², and X⁴ are CH. In certain of these embodiments, X³ is C^{a} (e.g., C—CH₃). In certain other embodiments, X³ is N. In certain of the foregoing embodiments (when X² and X⁴ are CH; or when X¹, X², and X⁴ are CH), A is C(═O)OH or P(═O)(OH)₂.

In some embodiments, the compound of Formula (I) is a compound of Formula (IA):

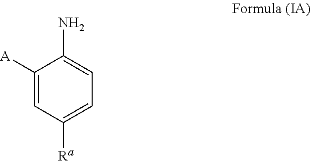

or a pharmaceutically acceptable salt thereof.

In some embodiments of Formula (IA), A is C(═O)OH. In some embodiments of Formula (IA), R^{a} is C₁₋₆ alkyl. In certain of these embodiments, R^{a} is C₁₋₃ alkyl. For example, R^{a} is methyl.

In some embodiments, the compound of Formula (I) is a compound of Formula (IB):

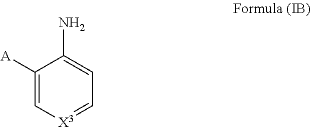

or a pharmaceutically acceptable salt thereof, wherein: X³ is CH or N.

In some embodiments of Formula (IB), A is P(═O)(OH)₂. In some embodiments of Formula (IB), X³ is N.

In some embodiments, the compound of Formula (I) is selected from the group consisting of:

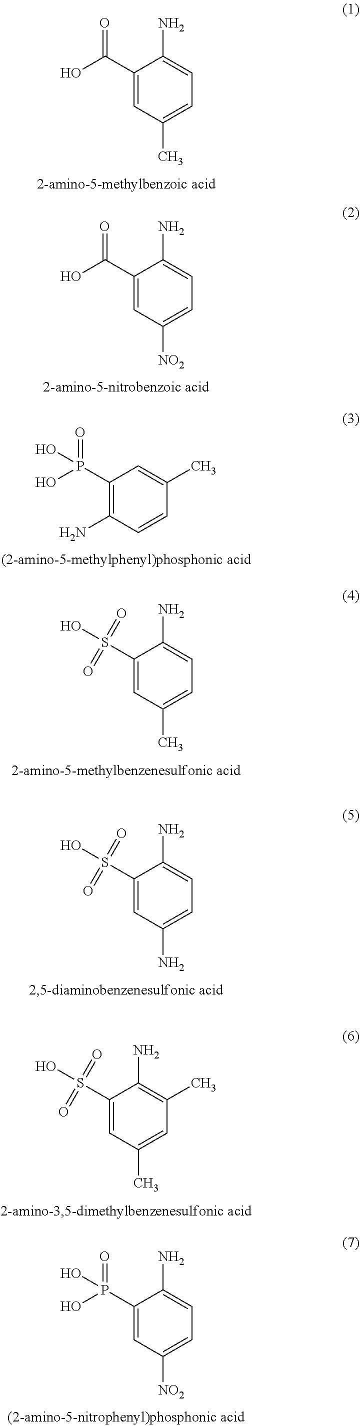

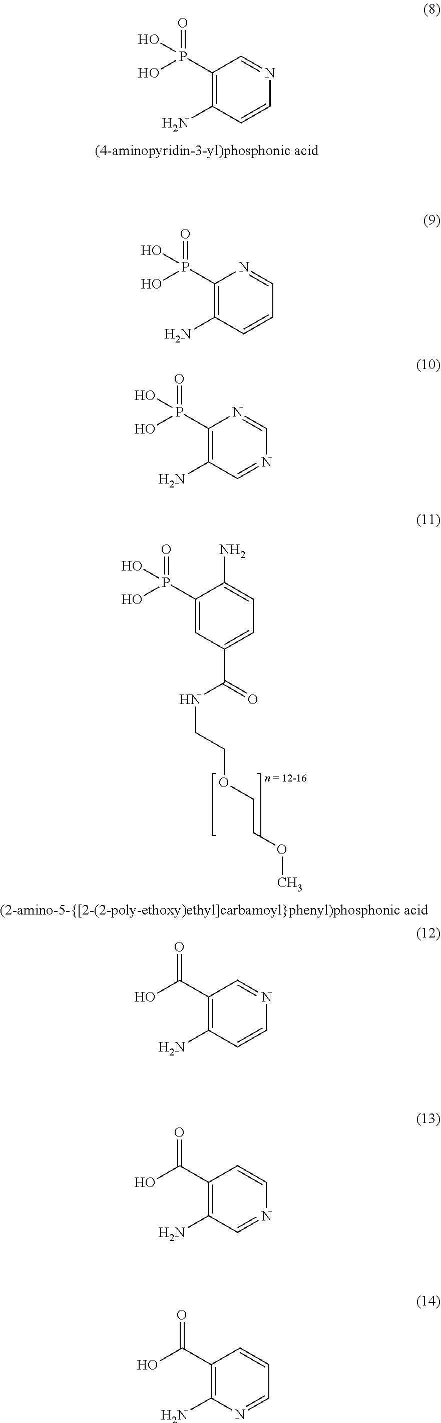

Compound (3) has previously been shown to catalytically break down the aminal and hemi-aminal adducts that form in RNA treated with formaldehyde, and is compatible with many RNA extraction and detection conditions. See e.g., Karmakar et al., “Organocatalytic removal of formaldehyde adducts from RNA and DNA bases,” Nature Chemistry, 7: 752-758 (2015); and US 2017/0283860A1, both of which are incorporated by reference herein in their entireties.

In some embodiments, the de-crosslinking agent is a compound of Formula (II):

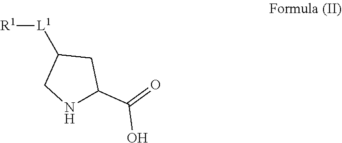

or a pharmaceutically acceptable salt thereof, wherein:
L¹ is selected from the group consisting of: —O—, —N(H)—, —N(C₁₋₃ alkyl)-, —S(O)₀₋₂—, —CH₂—, and a bond;
R¹ is selected from the group consisting of: H;
   C₁₋₆ alkyl;
   C₁₋₆ haloalkyl;
   C₆₋₁₀ aryl optionally substituted with 1-4 independently selected R^{b}; and
   heteroaryl of 5-10 ring atoms, wherein 1-4 ring atoms are heteroatoms each independently selected from the group consisting of: N, N(H), N(C₁₋₃ alkyl), O, and S, wherein the heteroaryl is optionally substituted with 1-4 independently selected R^{b}; and
each occurrence of R^{b} is independently selected from the group consisting of: halo, cyano, —OH, —NH₂, —NH(C₁₋₃ alkyl), —N(C₁₋₃ alkyl)₂, C₁₋₆ alkyl, C₁₋₆ haloalkyl, C₁₋₄ alkoxy, and C₁₋₄ haloalkoxy.

In some embodiments of Formula (II), -L¹-R¹ and the C(═O)OH group are cis to one another.

In some embodiments of Formula (II), -L¹-R¹ and the C(═O)OH group are trans to one another.

In some embodiments of Formula (II), the compound is a compound of Formula (II-a):

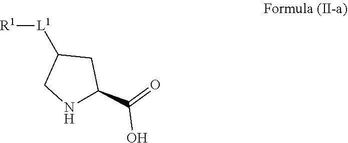

or a pharmaceutically acceptable salt thereof.

In some embodiments of Formula (II), the compound is a compound of Formula (II-a1):

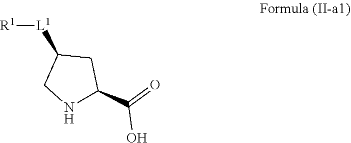

or a pharmaceutically acceptable salt thereof.

In some embodiments of Formula (II), the compound is a compound of Formula (II-a2):

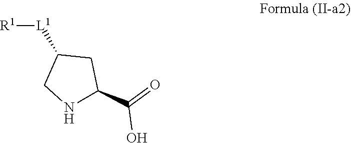

or a pharmaceutically acceptable salt thereof.

In some embodiments of Formulae (II), (II-a), (II-al), or (II-a2), L¹ is —O—.

In some embodiments of Formulae (II), (II-a), (II-al), or (II-a2), L¹ is —N(H)— or —N(C₁₋₃ alkyl)-. In certain of these embodiments, L¹ is —N(H)—.

In some embodiments of Formulae (II), (II-a), (II-a1), or (II-a2), R¹ is H.

In some embodiments of Formulae (II), (II-a), (II-al), or (II-a2), R¹ is heteroaryl of 5-10 ring atoms, wherein 1-4 ring atoms are heteroatoms each independently selected from the group consisting of: N, N(H), N(C₁₋₃ alkyl), O, and S, wherein the heteroaryl is optionally substituted with 1-4 independently selected R^{b}.

In certain of these embodiments, R¹ is heteroaryl of 5-6 ring atoms, wherein 1-4 ring atoms are heteroatoms each independently selected from the group consisting of: N, N(H), N(C₁₋₃ alkyl), O, and S, wherein the heteroaryl is optionally substituted with 1-2 independently selected R^{b}.

In certain of the foregoing embodiments, R¹ is heteroaryl of 6 ring atoms, wherein 1-2 ring atoms are ring nitrogen atoms, wherein the heteroaryl is optionally substituted with 1-2 independently selected R^{b}.

As a non-limiting example of the foregoing embodiments, can be pyridyl which is optionally substituted with 1-2 independently selected R^{b}. For example, R¹ can be 3-pyridyl which is optionally substituted with 1-2 independently selected R^{b} (e.g., unsubstituted 3-pyridyl). As another non-limiting example, R¹ can be 4-pyridyl which is optionally substituted with 1-2 R^{b} (e.g., unsubstituted 4-pyridyl).

In some embodiments of Formula (II), the compound is a compound of Formula (II-al); L¹ is —O—; and is heteroaryl of 6 ring atoms, wherein 1-2 ring atoms are ring nitrogen atoms, wherein the heteroaryl is optionally substituted with 1-2 independently selected R^{b}. In certain of these embodiments, R¹ is pyridyl which is optionally substituted with 1-2 independently selected R^{b}. For example, R¹ can be 3-pyridyl which is optionally substituted with 1-2 independently selected R^{b} (e.g., unsubstituted 3-pyridyl). As another non-limiting example, R¹ can be 4-pyridyl which is optionally substituted with 1-2 R^{b} (e.g., unsubstituted 4-pyridyl).

In some embodiments of Formula (II), the compound is a compound of Formula (II-a2); L¹ is —O—, —N(H)—, or —N(C₁₋₃ alkyl)-; and R¹ is H.

In some embodiments, the compound of Formula (II) is selected from the group consisting of:

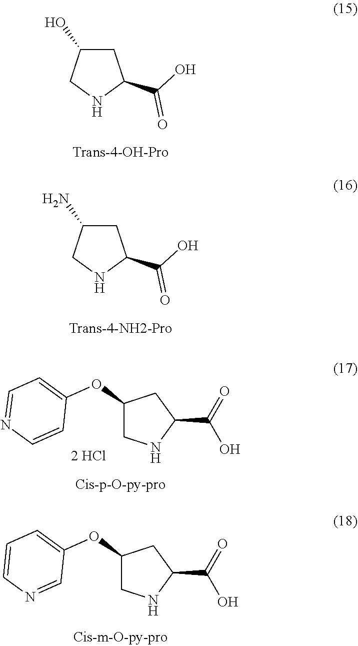 [SMILES: O=C(O)[C@@H]1C[C@@H](O)CN1 ; N[C@H]1CN[C@H](C(=O)O)C1 ; O=C(O)[C@@H]1C[C@H](Oc2ccncc2)CN1 ; O=C(O)[C@@H]1C[C@H](Oc2cccnc2)CN1]

or a pharmaceutically acceptable salt thereof.

In some embodiments, the de-crosslinking agent can comprise compound (1), compound (2), compound (3), compound (4), compound (5), compound (6), compound (7), compound (8), compound (9), compound (10), compound (11), or a combination thereof. In some embodiments, the de-crosslinking agent can comprise compound (1), compound (2), compound (4), compound (5), compound (6), compound (7), compound (8), compound (9), compound (10), compound (11), or a combination thereof. In some embodiments, the de-crosslinking agent can comprise compound (1), compound (2), compound (3), compound (4), compound (5), compound (6), compound (7), compound (8), compound (9), compound (10), compound (11), compound (12), compound (13), compound (14), or a combination thereof. In some embodiments, the de-crosslinking agent can comprise compound (1), compound (2), compound (4), compound (5), compound (6), compound (7), compound (8), compound (9), compound (10), compound (11), compound (12), compound (13), compound (14), or a combination thereof. In some embodiments, the de-crosslinking agent can comprise compound (1), compound (2), compound (3), compound (4), compound (5), compound (6), compound (7), compound (8), compound (9), compound (10), compound (11), compound (12), compound (13), compound (14), compound (15), compound (16), compound (17), compound (18), or a combination thereof. In some embodiments, the de-crosslinking agent can comprise compound (1), compound (2), compound (4), compound (5), compound (6), compound (7), compound (8), compound (9), compound (10), compound (11), compound (12), compound (13), compound (14), compound (15), compound (16), compound (17), compound (18), or a combination thereof. In some embodiments, the de-crosslinking agent can comprise compound (8), compound (9), compound (10), or a combination thereof.

In some embodiments, the de-crosslinking agent can be selected from the group consisting of compound (1), compound (2), compound (3), compound (4), compound (5), compound (6), compound (7), compound (8), compound (9), compound (10), compound (11), and a combination thereof. In some embodiments, the de-crosslinking agent can be selected from the group consisting of compound (1), compound (2), compound (4), compound (5), compound (6), compound (7), compound (8), compound (9), compound (10), compound (11), and a combination thereof. In some embodiments, the de-crosslinking agent can be selected from the group consisting of compound (1), compound (2), compound (3), compound (4), compound (5), compound (6), compound (7), compound (8), compound (9), compound (10), compound (11), compound (12), compound (13), compound (14), and a combination thereof. In some embodiments, the de-crosslinking agent can be selected from the group consisting of compound (1), compound (2), compound (4), compound (5), compound (6), compound (7), compound (8), compound (9), compound (10), compound (11), compound (12), compound (13), compound (14), and a combination thereof. In some embodiments, the de-crosslinking agent can be selected from the group consisting of compound (1), compound (2), compound (3), compound (4), compound (5), compound (6), compound (7), compound (8), compound (9), compound (10), compound (11), compound (12), compound (13), compound (14), compound (15), compound (16), compound (17), compound (18), and a combination thereof. In some embodiments, the de-crosslinking agent can be selected from the group consisting of compound (1), compound (2), compound (4), compound (5), compound (6), compound (7), compound (8), compound (9), compound (10), compound (11), compound (12), compound (13), compound (14), compound (15), compound (16), compound (17), compound (18), and a combination thereof. In some embodiments, the de-crosslinking agent can be selected from the group consisting of compound (8), compound (9), compound (10), and a combination thereof. In some embodiments, the de-crosslinking agent is compound (1), either alone or in combination with one or more of the aforementioned compounds. In some embodiments, the de-crosslinking agent is compound (1).

A de-crosslinking agent can be contacted with (e.g., applied to) a biological sample at any appropriate concentration. An appropriate concentration may depend on factors such as tissue type, fixation reagent used, and degree of crosslinking in the biological sample. In some embodiments, a de-crosslinking agent can be contacted with (e.g., applied to) a biological sample in a solution or suspension with a concentration of about 10 mM to about 500 mM (e.g., about 10 mM to about 100 mM, about 10 mM to about 200 mM, about 10 mM to about 300 mM, about 10 mM to about 400 mM, about 100 mM to about 200 mM, about 100 mM to about 300 mM, about 100 mM to about 400 mM, about 100 mM to about 500 mM, about 200 mM to about 300 mM, about 200 mM to about 400 mM, about 200 mM to about 500 mM, about 300 mM to about 400 mM, about 300 mM to about 500 mM, or about 400 mM to about 500 mM). In some embodiments, a de-crosslinking agent can be contacted with (e.g., applied to) a biological sample in a solution or suspension with a concentration of about 10 mM to about 100 mM (e.g., about 10 mM to about 20 mM, about 10 mM to about 30 mM, about 10 mM to about 40 mM, about 10 mM to about 50 mM, about 10 mM to about 60 mM, about 10 mM to about 70 mM, about 10 mM to about 80 mM, about 10 mM to about 90 mM, about 20 mM to about 30 mM, about 20 mM to about 40 mM, about 20 mM to about 50 mM, about 20 mM to about 60 mM, about 20 mM to about 70 mM, about 20 mM to about 80 mM, about 20 mM to about 90 mM, about 20 mM to about 100 mM, about 30 mM to about 40 mM, about 30 mM to about 50 mM, about 30 mM to about 60 mM, about 30 mM to about 70 mM, about 30 mM to about 80 mM, about 30 mM to about 90 mM, about 30 mM to about 100 mM, about 40 mM to about 50 mM, about 40 mM to about 60 mM, about 40 mM to about 70 mM, about 40 mM to about 80 mM, about 40 mM to about 90 mM, about 40 mM to about 100 mM, about 50 mM to about 60 mM, about 50 mM to about 70 mM, about 50 mM to about 80 mM, about 50 mM to about 90 mM, about 50 mM to about 100 mM, about 60 mM to about 70 mM, about 60 mM to about 80 mM, about 60 mM to about 90 mM, about 60 mM to about 100 mM, about 70 mM to about 80 mM, about 70 mM to about 90 mM, about 70 mM to about 100 mM, about 80 mM to about 90 mM, about 80 mM to about 100 mM, or about 90 mM to about 100 mM) of the de-crosslinking agent. In some embodiments, a de-crosslinking agent can be contacted with (e.g., applied to) a biological sample in a solution or suspension with a concentration of about 30 mM to about 70 mM of the de-crosslinking agent. In some embodiments, a de-crosslinking agent can be contacted with (e.g., applied to) a biological sample in a solution or suspension with a concentration of about 40 mM to about 60 mM of the de-crosslinking agent. In some embodiments, a de-crosslinking agent can be contacted with (e.g., applied to) a biological sample in a solution or suspension with a concentration of about 50 mM of the de-crosslinking agent.

A de-crosslinking agent can be delivered to a biological sample using any appropriate method. In some embodiments, a de-crosslinking agent can be delivered as a solution or a suspension. In some embodiments, a de-crosslinking agent can be delivered as a solution or a suspension in a buffer. In some embodiments, the buffer is Tris, TE, PBS, HEPES, or MES. In some embodiments, the buffer is Tris. In some embodiments, the buffer is a TE buffer. A buffer can have any appropriate concentration. For example, in some embodiments, a buffer can have a concentration of about 5 mM to about 60 mM (e.g., about 10 mM to about 50 mM, about 20 mM to about 40 mM, or about 30 mM).

A biological sample (e.g., tissue section) can be incubated while the de-crosslinking agent is contacted with (e.g., applied to) the biological sample. In some embodiments, the biological sample (e.g., tissue section) can be incubated between about 25° C. and about 100° C. In some embodiments, the biological sample (e.g., tissue section) can be incubated between about 25° C. and about 40° C., about 37° C. and about 60° C., about 45° C. and about 95° C., about 50° C. and about 90° C., about 55° C. and about 85° C., about 60° C. and about 80° C., about 65° C. and about 75° C. In some embodiments, the biological sample (e.g., tissue section) can be incubated at about 70° C.

The de-crosslinking agent can be contacted to the biological sample for a time sufficient to de-crosslink some or all of the crosslinked nucleic acids and/or proteins in the biological sample (e.g., tissue section). In some embodiments, the de-crosslinking agent can be contacted to the biological sample (e.g., tissue section) for between 1 minute and 1 day (e.g., between 1 minute and 1 hour, 1 minute and 2 hours, 1 minute and 4 hours, 1 minute and 6 hours, 1 minute and 12 hours, 1 minute and 18 hours, 1 hour and 2 hours, 1 hour and 4 hours, 1 hour and 6 hours, 1 hour and 12 hours, 1 hour and 18 hours, 1 hour and 1 day, 2 hours and 4 hours, 2 hours and 6 hours, 2 hours and 12 hours, 2 hours and 18 hours, 2 hours and 1 day, 4 hours and 6 hours, 4 hours and 12 hours, 4 hours and 18 hours, 4 hours and 1 day, 6 hours and 12 hours, 6 hours and 18 hours, 6 hours and 1 day, 12 hours and 18 hours, 12 hours and 1 day, or 18 hours and 1 day). In some embodiments, an incubation temperature and a contact time can be related. Without being bound by any particular theory, it is believed that if a higher temperature is used, a shorter contact time may be sufficient (e.g., 70° C. to 80° C. for 1 hour), while if a lower temperature is used, a longer contact time may be beneficial (e.g., 37° C. for 1 day). However, in some cases, both a low temperature and a shorter contact time may be sufficient (e.g., 20° C. to 28° C. for 90 minutes). In some embodiments, the de-crosslinking agent can be contacted to the biological sample (e.g., tissue section) for between 1 hour and 120 minutes (e.g, between 1 minute and 110 minutes, 1 minute and 100 minutes, 1 minute and 90 minutes, 1 minute and 80 minutes, 1 minute and 70 minutes, 10 minutes and 120 minutes, 20 minutes and 120 minutes, 30 minutes and 120 minutes, 40 minutes and 120 minutes, 50 minutes and 120 minutes. In some embodiments, the de-crosslinking agent can be applied to the biological sample (e.g., tissue section) for about 10 minutes, about 20, 30, 40, 50, 60, 70, 80, 90, 110, or about 120 minutes. In some embodiments, the de-crosslinking agent can be contacted to the biological sample (e.g., tissue section) for approximately 60 minutes.

A de-crosslinking agent can be contacted with (e.g., applied to) the biological sample (e.g., tissue section) in any number of ways. In some embodiments, a de-crosslinking agent is in solution or a suspension. In some embodiments, the biological sample (e.g., tissue section) is soaked in a solution or suspension comprising the de-crosslinking agent. In some embodiments, the de-crosslinking agent is sprayed onto the biological sample (e.g., tissue section) (e.g., as a solution or suspension). In some embodiments, the de-crosslinking agent is supplied to the biological sample (e.g., tissue section) via a microfluidic system (e.g., as a solution or suspension). In some embodiments, a de-crosslinking agent is pipetted or otherwise aliquoted onto the biological sample. In some embodiments, the biological sample (e.g., tissue section) is dipped into a solution or suspension of a de-crosslinking agent, wherein excess solution or suspension is removed from the biological sample (e.g., tissue section). In some embodiments, a de-crosslinking agent can be delivered to the biological sample (e.g., tissue section) via a hydrogel, wherein the hydrogel is contacted with the biological sample (e.g., tissue section). Application of a de-crosslinking agent can occur in other ways known in the art.

(d) Permeabilization

In some embodiments, a biological sample (e.g., tissue section) is permeabilized (e.g., undergoes permeabilization). See, e.g., Section (1)(d)(ii)(13) of WO 2020/176788 and/or U.S. Patent Application Publication No. 2020/0277663. In some embodiments, a biological sample (e.g., tissue section) is permeabilized after delivery to or application of a de-crosslinking agent. In some embodiments, the permeabilization comprises harsher conditions than the optional pretreatment step. In some embodiments, the permeabilization comprises applying one or more permeabilization reagents to the biological sample. In some embodiments, a permeabilization reagent can comprise a protease. In some embodiments, the protease comprises pepsin. In some embodiments, the protease comprises proteinase K. In some embodiments, the protease comprises ArcticZyme Proteinase. In some embodiments, the protease is provided in a solution of hydrochloric acid.

The permeabilization reagent(s) can be applied to the biological sample (e.g., tissue section) in any number of ways. In some embodiments, the permeabilization reagents can be in solution or suspension. In some embodiments, the biological sample (e.g., tissue section) is soaked in a solution or suspension of the permeabilization reagent(s). In some embodiments, the permeabilization reagents are sprayed onto the biological sample (e.g., tissue section) (e.g., as a solution or suspension). In some embodiments, the permeabilization reagents are supplied to the biological sample (e.g., tissue section) via a microfluidic system (e.g., as a solution or suspension). In some embodiments, the biological sample (e.g., tissue section) can be dipped into a solution or suspension comprising the permeabilization reagent(s), wherein excess permeabilization reagent is removed from the biological sample (e.g., tissue section). In some embodiments, the permeabilization reagent(s) are delivered to the biological sample (e.g., tissue section) via a hydrogel, wherein the hydrogel is contacted with the biological sample (e.g., tissue section). Application of the permeabilization reagent can occur in other ways known in the art.

The permeabilization reagent(s) can be contacted to the biological sample (e.g., tissue section) for a time sufficient to permeabilize biological sample (e.g., tissue section) so that analytes can migrate out of the biological sample (e.g., tissue section). In some embodiments, the permeabilization reagent(s) can be contacted to the biological sample (e.g., tissue section) for between 1 minute and 120 minutes. In some embodiments, the permeabilization reagent(s) can be applied to the biological sample (e.g., tissue section) between about 1 minute and 90 minutes, 1 minute and 80 minutes, 1 minute and 70 minutes, 1 minute and 60 minutes, 1 minute and about 55 minutes, about 1 minute and about 50 minutes, about 1 minute and about 45 minutes, about 1 minute and about 40 minutes, about 1 minute and about 35 minutes, about 1 minute and about 30 minutes, about 1 minute about 5 minutes and about 60 minutes, about 10 minutes and about 60 minutes, about 10 minutes and about 55 minutes, about 10 minutes and about 50 minutes, about 10 minutes and about 45 minutes, about 10 minutes and about 40 minutes, about 15 minutes and about 45 minutes, about 20 minutes and about 40 minutes, about 25 minutes and about 35 minutes. In some embodiments, the permeabilization reagent(s) can be contacted to the biological sample (e.g., tissue section) for approximately 30 minutes.

The biological sample (e.g., tissue section) can be incubated with the permeabilization reagent(s). In some embodiments, the biological sample (e.g., tissue section) can be incubated between about 16° C. and about 56° C. (e.g., between about 30° C. and 45° C., or between about 35° C. and about 40° C.). In some embodiments, the biological sample can be at about 16° C., 18° C., 20° C., 22° C., 24° C., 26° C., 28° C., 30° C., 31° C., 32° C., 33° C., 34° C., 35° C., 36° C., 37° C., 38° C., 39° C., 40° C., 41° C., 42° C., 43° C., 44° C., 45° C., 46° C., 48° C., 50° C., 52° C., 54° C., or 56° C. In some embodiments, the biological sample (e.g., tissue section) can be incubated at about 37° C.

(e) Spatial Analysis

In some embodiments, the methods provided herein for use with a fixed biological sample (e.g., preparation of a fixed biological sample, pretreatment of a fixed biological sample, de-crosslinking of a fixed biological sample, permeabilization of a de-crosslinked biological sample, or a combination thereof) can be used with any of the spatial analysis methods described herein; see also, e.g., Section (II)(e)-(g) of WO 2020/176788 and/or U.S. Patent Application Publication No. 2020/0277663. In some embodiments, spatial analysis of a fixed biological sample using the steps described herein (e.g., preparation of a fixed biological sample, pretreatment of a fixed biological sample, de-crosslinking of a fixed biological sample, permeabilization of a de-crosslinked biological sample, or a combination thereof) can yield more efficient and/or more accurate results than a similar spatial analysis workflow when one or more of the steps (e.g., preparation of a fixed biological sample, pretreatment of a fixed biological sample, de-crosslinking of a fixed biological sample, permeabilization of a de-crosslinked biological sample, or a combination thereof) are not performed. In some embodiments, the fixed biological sample includes aminal crosslinks. In some such embodiments, aminal crosslinks can be reversed by a de-crosslinking agent, such as those described herein.

In some embodiments, provided herein are methods of producing a de-crosslinked biological sample (e.g., for spatial analysis), using any of the methods described herein. The steps can be carried out in any appropriate order. For example, in some cases, a fixed biological sample (e.g., tissue section) can be de-crosslinked before being contacted with an array for spatial analysis, while in other cases, a fixed biological sample (e.g., tissue section) can be de-crosslinked after being contacted for spatial analysis. Accordingly, in some embodiments, the method can include: (a) contacting a fixed biological sample with a substrate comprising a plurality of capture probes, wherein a capture probe of the plurality of capture probes comprises a capture domain; and (b) contacting the fixed biological sample with a de-crosslinking agent (e.g., any of the de-crosslinking agents described herein) thereby producing the de-crosslinked biological sample. In some embodiments, the methods can include (a) contacting a fixed biological sample with a de-crosslinking agent, thereby producing the de-crosslinked biological sample; and (b) contacting the de-crosslinked biological sample with a substrate comprising a plurality of capture probes, wherein a capture probe of the plurality of capture probes comprises a capture domain. In some embodiments, contacting a fixed biological sample with a de-crosslinking agent includes applying the de-crosslinking agent to the biological sample using any of the methods described herein, such as soaking, pipetting, spraying, or dipping.

In some embodiments, the methods can optionally include a step of deparaffinizing the fixed biological sample, thereby producing a de-paraffinized fixed biological sample, and optionally rehydrating the de-paraffinized fixed biological sample, for example, when the fixed biological sample is an FFPE sample. Deparaffinizing can be performed using any of the methods or protocols described herein, see, for example, section (I)(d)(ii)(3) of WO 2020/176788 and/or U.S. Patent Application Publication No. 2020/0277663. Typically, deparaffinizing is performed before contacting the fixed biological sample (e.g., tissue section) with the de-crosslinking agent. Deparaffinizing the fixed biological sample (e.g., tissue section) can be carried out either before or after the fixed biological sample is contacted with the substrate.

In some embodiments, the methods can optionally include a step pretreating the fixed biological sample or de-paraffinized fixed biological sample. Without being bound by any particular theory, it is believed that deparaffinizing and/or pretreating a fixed biological sample (e.g., tissue section) can allow for greater penetration of a de-crosslinking agent into the fixed biological sample. Pretreating can be performed using any of the methods or protocols described herein (e.g., in section (b), above). Typically, pretreating is performed before contacting the fixed biological sample with the de-crosslinking agent. If deparaffinizing is performed, pretreating generally follows deparaffinizing. Pretreating the fixed biological sample can be performed either before or after the fixed biological sample (e.g., tissue section) is contacted with the substrate.

In some embodiments, the methods can optionally include a step of permeabilizing the de-crosslinked biological sample. Permeabilizing can be performed using any of the methods or protocols described herein, see, for example, sections (I)(d)(ii)(13) of WO 2020/176788 and/or U.S. Patent Application Publication No. 2020/0277663 and/or section (d) above. Typically, permeabilizing the de-crosslinked biological sample (e.g., tissue section) is performed after the de-crosslinked biological sample is contacted with the substrate.

In some embodiments, the methods can optionally include staining and/or imaging of the fixed biological sample (e.g., tissue section), of the de-crosslinked biological sample (e.g., tissue section), or both. A stain can be any appropriate stain, such as a histological stain (e.g., hematoxylin and eosin) or an immunological stain (e.g., an immunofluorescent stain), or any other stain described herein or known in the art. Staining and/or imaging can be carried out according to known methods. Typically, staining and/or imaging is performed after the fixed biological sample (e.g., tissue section) or de-crosslinked biological sample (e.g., tissue section) is contacted with the substrate but before the de-crosslinked biological sample is permeabilized, if permeabilization is performed.

The capture probe of the plurality of capture probes of the substrate can have any appropriate features, such as any of those described in section (II)(b) of WO 2020/176788 and/or U.S. Patent Application Publication No. 2020/0277663. In some embodiments, the capture probe of the plurality of capture probes can include a spatial barcode.

In some embodiments, further provided herein are methods of determining a location of an analyte in a fixed biological sample. The methods can include (i) preparing a de-crosslinked biological sample according to any of the methods described herein.

In some embodiments, the analyte comprises a nucleic acid analyte, where the capture domain of the capture probe binds specifically to the nucleic acid analyte. In some such embodiments, the methods can further include (ii) determining (I) a sequence corresponding to the nucleic acid analyte or a complement thereof, and (II) a sequence corresponding to the spatial barcode of the capture probe or a complement thereof, and using the determined sequences of (I) and (II) to determine the location of the nucleic acid analyte in the de-crosslinked biological sample. A nucleic acid analyte can be any of the nucleic acid analytes described herein, such as DNA (e.g., gDNA) and/or RNA (e.g., mRNA). Determining of the sequences of (I) and (II) can be performed using any appropriate method. In some embodiments, sequencing (e.g., high-throughput sequencing) can be used.

In some embodiments, the analyte comprises a protein analyte. In some such embodiments, the methods can further include (ii) contacting the de-crosslinked biological sample with a plurality of analyte capture agents, wherein an analyte capture agent of the plurality of analyte capture agents includes (1) an analyte binding moiety that binds specifically to a protein analyte from the de-crosslinked biological sample; (2) a capture agent barcode domain comprising an analyte binding moiety barcode and an analyte capture sequence, wherein the analyte capture sequence binds specifically to the capture domain of the capture probe; and (iii) determining (I) a sequence corresponding to the analyte binding moiety barcode, and (II) a sequence corresponding to the spatial barcode of the capture probe or a complement thereof, and using the determined sequences of (I) and (II) to determine the location of the protein analyte in the fixed biological sample. An analyte capture agent can be any appropriate analyte capture agent, see, e.g., section (II)(b)(ix) of WO 2020/176788 and/or section (II)(b)(viii) of U.S. Patent Application Publication No. 2020/0277663. For example, in some embodiments, an analyte binding moiety can be an antibody or antigen-binding fragment thereof. A protein analyte can be any appropriate analyte, such as an intracellular protein, an extracellular protein, and/or a cell surface protein. Determining of the sequences of (I) and (II) can be performed using any appropriate method. In some embodiments, sequencing (e.g., high-throughput sequencing) can be used.

It will be understood that while the determination of the location of a single analyte is described, the location of multiple analytes (and/or types of analytes) can be performed by analyzing additional capture probes of the plurality of capture probes and/or additional pluralities of capture probes.

(f) Kits

Also provided herein are kits that can be used for de-crosslinking of a fixed biological sample, and optionally, subsequent spatial analysis of a de-crosslinked biological sample. In some cases, such a kit can be used to practice any of the de-crosslinking and/or spatial analysis methods described herein. In some embodiments, such a kit can include a substrate comprising a plurality of capture probes, wherein a capture probe comprise a spatial barcode and a capture domain and a reagent comprising a compound of Formula (I). In some embodiments, such a kit can include a substrate comprising a plurality of capture probes, wherein a capture probe comprise a spatial barcode and a capture domain and a reagent comprising a compound of Formula (II). In some embodiments, such a kit can include a substrate comprising a plurality of capture probes, wherein a capture probe comprise a spatial barcode and a capture domain and a reagent comprising one or more of compounds (1)-(18). In some embodiments, the kit includes compound (1). A substrate can be any appropriate substrate, including those described in Section (II)(c) of WO 2020/176788 and/or U.S. Patent Application Publication No. 2020/0277663. A capture probe, in some cases, can be a capture probe as described in section (II)(b) of WO 2020/176788 and/or U.S. Patent Application Publication No. 2020/0277663. In some embodiments, the capture probe can include a spatial barcode.

A kit as described herein can include any other appropriate reagents or components for carrying out the methods described herein. Non-limiting examples of such reagents or components include one or more polymerase enzymes, one or more wash buffers, one or more reaction buffers, or a combination thereof. For example, an polymerase enzyme can include an RNA-dependent DNA polymerase (e.g., a reverse transcriptase), a DNA polymerase, a terminal deoxynucleotidyl transferase, or two or more thereof. As another example, a wash buffer can be used to remove nucleic acids and/or analyte capture agents not specifically bound to the capture probes. As yet another example a reaction buffer can include a buffering agent, and/or a cofactor useful in reverse transcription and/or nucleic acid amplification steps. In some embodiments, a reaction buffer can include an enzyme, such as an enzyme different from a first enzyme in the kit, such as a different polymerase or a ligase. See, e.g., Sections (I)(b)(xiii) and (II)(a) of (II)(b) of WO 2020/176788 and/or U.S. Patent Application Publication No. 2020/0277663.

### EXAMPLES

### Example 1—De-Crosslinking Workflow

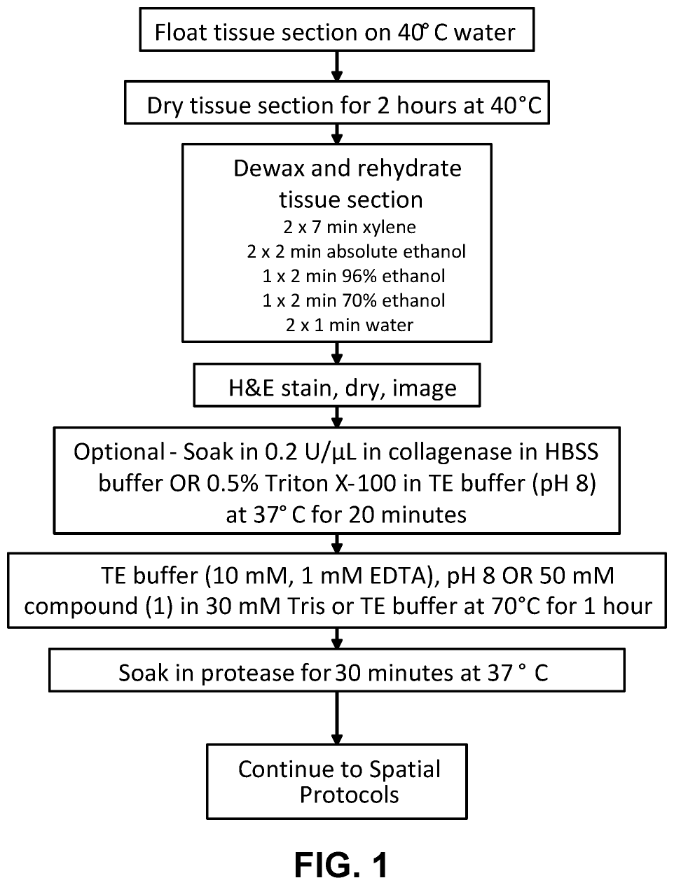

An exemplary de-crosslinking workflow is shown in FIG. 1. Paraffin-embedded, formalin-fixed tissue were sectioned, and the sections were immersed in 40° C. water for attaching the tissue section on a slide. The sections were dried for 2 hours at 40° C. The tissue section was dewaxed and rehydrated using a protocol of 2×7 minutes in xylene, 2×2 minutes in absolute ethanol, 1×2 minutes in 96% ethanol, 1×2 minutes in 70% ethanol, 2×1 minute in water. The tissue section was stained using hematoxylin and eosin, dried, and imaged using bright-field microscopy. After imaging, the tissue section can be optionally pre-treated with 0.2 U/μL collagenase in HBSS buffer or 0.5% Triton X-100 in TE buffer, pH 8, for ˜20 minutes at 37° C. After pre-treatment, the tissue section was then de-crosslinked using 50 mM 2-amino-5-methylbenzoic acid (CATJ1) in 30 mM Tris or TE buffer or TE buffer, 10 mM Tris, 1 mM EDTA, pH 8, for ˜1 hour at 70° C. After de-crosslinking, the tissue section was treated with pepsin or proteinase K for ˜30 minutes at 37° C. The paraffin-embedded, formalin-fixed tissue section is ready to be used in spatial analysis and analyte capture protocols.

### Example 2—Spatial Gene Expression Analysis of FFPE Mouse Spleen Tissue Sections Using Spatial Analysis

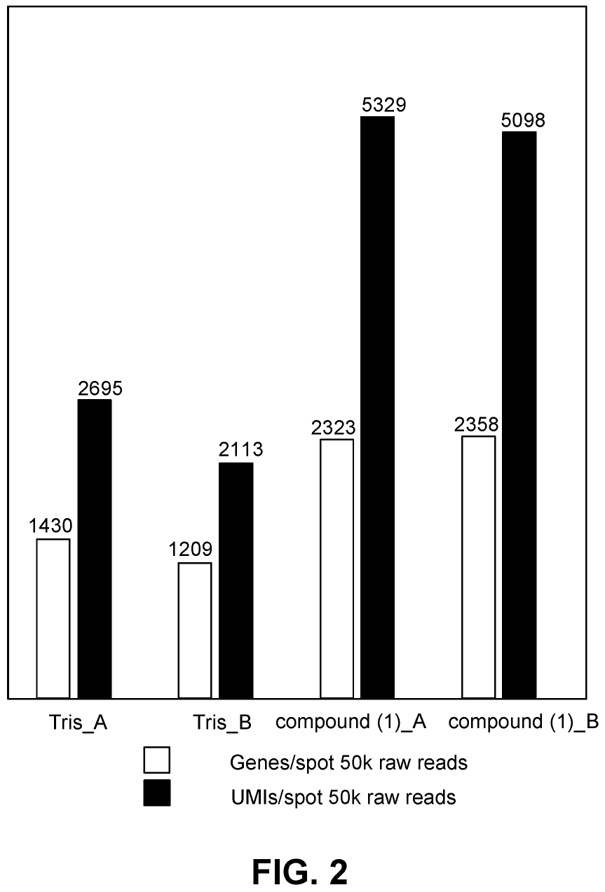

In order to determine whether analytes from a previously fixed biological sample can be de-crosslinked and captured on a spatial array for spatial gene expression analysis, a de-crosslinking workflow was performed using a de-crosslinking agent described herein, and compared to treatment with a TE buffer. Specifically, a de-crosslinking workflow was performed as described in Example 1 using 50 mM 2-amino-5-methylbenzoic acid in 30 mM Tris buffer at pH 8 at 70° C. for 1 hour and compared to a TE buffer comprising 10 mM Tris and 1 mM EDTA at pH 8 at 70° C. for 1 hour. The 2-amino-5-methylbenzoic acid and TE de-crosslinking were followed by pepsin permeabilization and spatial analysis methods as described herein. Spatial gene expression analysis was performed and the number of genes per spot with 50K raw reads and the number of unique molecular identifiers (UMIs) per spot with 50K raw reads was determined. In FIG. 2, the Tris A and Tris B data sets are duplicates of a single protocol and show the number of genes per spot and the number of unique molecular identifiers per spot for a de-crosslinking protocol using TE buffer (10 mM Tris, 1 mM EDTA, pH 8). The compound (1)_A and compound (1)_B data sets are duplicates of a single protocol and show the number of genes per spot and the number of unique molecular identifiers (UMIs) per spot for a de-crosslinking protocol using 2-amino-5-methylbenzoic acid (compound (1)). FIG. 2 shows that both the number of genes per spot as well as the number of UMIs per spot are greater for the 2-amino-5-methylbenzoic acid (compound (1)_A and compound (1)_B) de-crosslinked duplicate samples than for the TE (Tris A and Tris B) duplicate sample treatment, indicating improved assay sensitivity in that more analytes were able to move out of the biological sample and be captured by the capture probes on the spatial array.

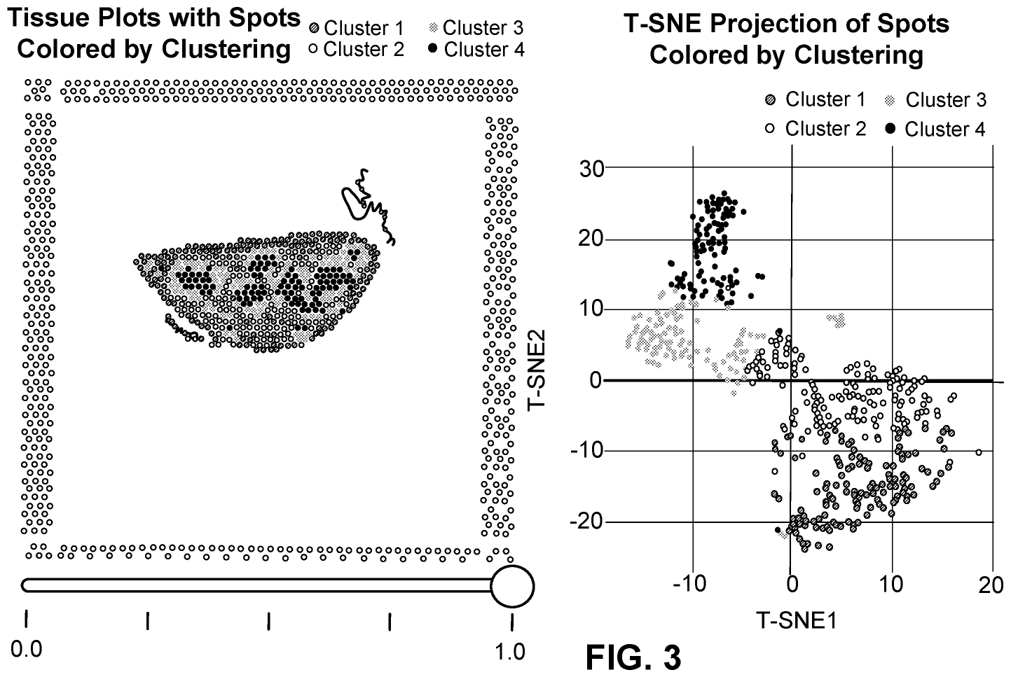

FIG. 3 demonstrates that the de-crosslinking methods described herein are compatible with identification of morphological features within a biological sample. Spatial gene expression of FFPE mouse spleen tissue sections can be further analyzed by t-SNE or other dimensionality reduction algorithms, wherein features or spots can be grouped and colored by clustering on a t-SNE plot and mapped back onto an image of the tissue analyzed. The colored features can indicate clusters of genes expressed at that particular feature location, and the clusters can indicate morphological features within a biological sample. In FIG. 3, the data clusters identify distinct morphological features of the tissue section after decrosslinking with 2-amino-5-methylbenzoic acid (compound (1)).

### Example 3—De-Crosslinking with Various Catalysts

Additional catalysts of Formulas (I) and (II) were evaluated for the ability to de-crosslink fixed cells. For each of the experiments below, after the bulk un-fixing treatment of the PFA-fixed cells, the resulting samples were centrifuged 5 minutes at 500 g, 4° C., and the supernatant and pellet fractions were collected separately. RNA isolation from collected pellets and supernatants was performed using RNeasy Plus Mini Kit (Qiagen, Cat #_74134) and RNeasy MinElute Cleanup Kit (Qiagen, Cat #74204), respectively. Isolated RNA was quantified using Qubit™ RNA HS Assay Kit (Invitrogen, Cat #Q32855) and Agilent RNA ScreenTape System (Agilent Technologies). While these experiments were carried out on cells in suspension, it is believed that the decrosslinking agents will perform their function in any fixed cell, including in tissue samples (e.g., tissue sections for spatial analysis).

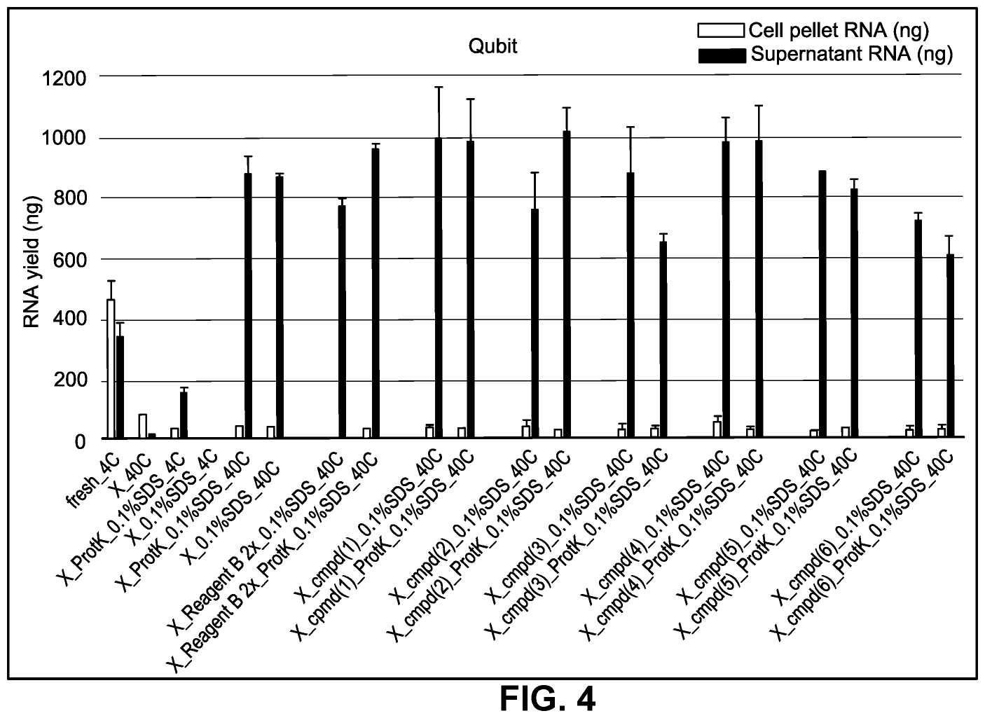

In FIG. 4, de-crosslinking was evaluated by determining the amount of RNA recovered from the cell pellet and the supernatant of peripheral blood mononuclear cells (PBMCs). PBMCs were fixed with 4% PFA for 20 minutes at 4° C. To de-crosslink, the cells were treated with 0.1% SDS in 30 mM Tris (pH 6.8) for 2 hours at 40° C., with or without addition of proteinase, in the presence or absence of compounds (1)-(6) (20 mM, 2 hours at 4° C.), or Reagent B from Cell Data Sciences. Compared to control treatments, cells treated with compounds (1)-(6) generally have increased RNA recovery from the cell pellet and/or cell supernatant. From fresh cells, 523 ng of RNA was isolated from the cell pellet.

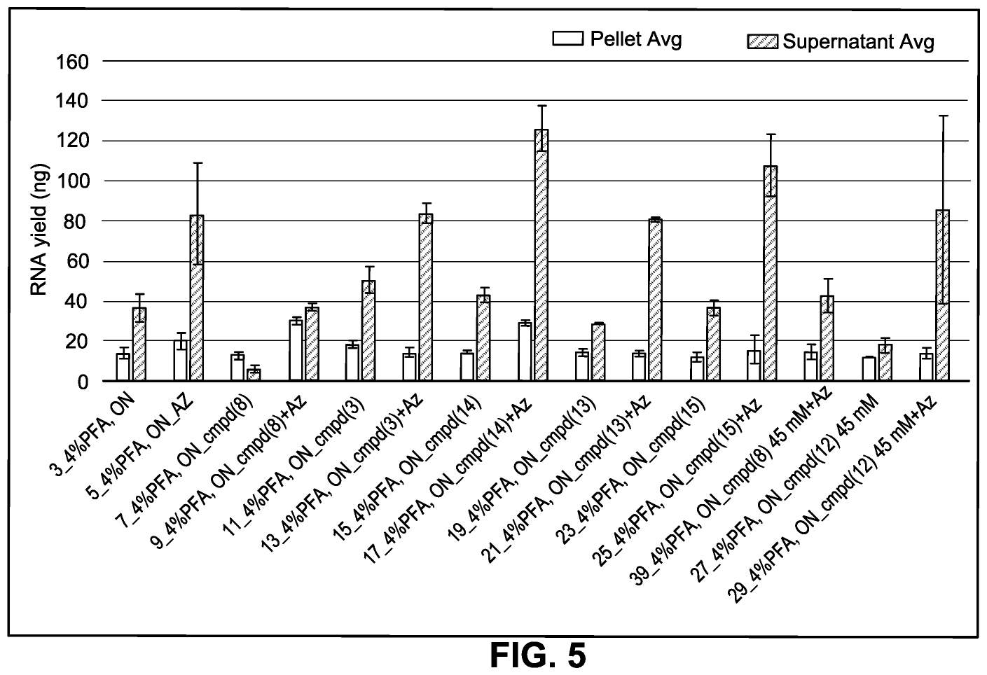

In FIG. 5, de-crosslinking was evaluated by determining the amount of RNA recovered from the cell pellet and the supernatant of Jurkat cells. Jurkat cells were fixed by treating with 4% PFA for 16 hours at 4° C. (1:10 cells to PFA by volume). The experimental cells were treated with one of compounds (3), (8), (12), (13), (14), or (15) (100 mM or as otherwise indicated, 53° for 45 minutes and then 85° C. for 5 minutes), in the presence or absence of ArcticZyme proteinse (10 U/mL). Compared to controls, cells treated with one of compounds (3), (8), (12), (13), (14), or (15) generally have increased RNA recovery from the cell pellet and/or cell supernatant.

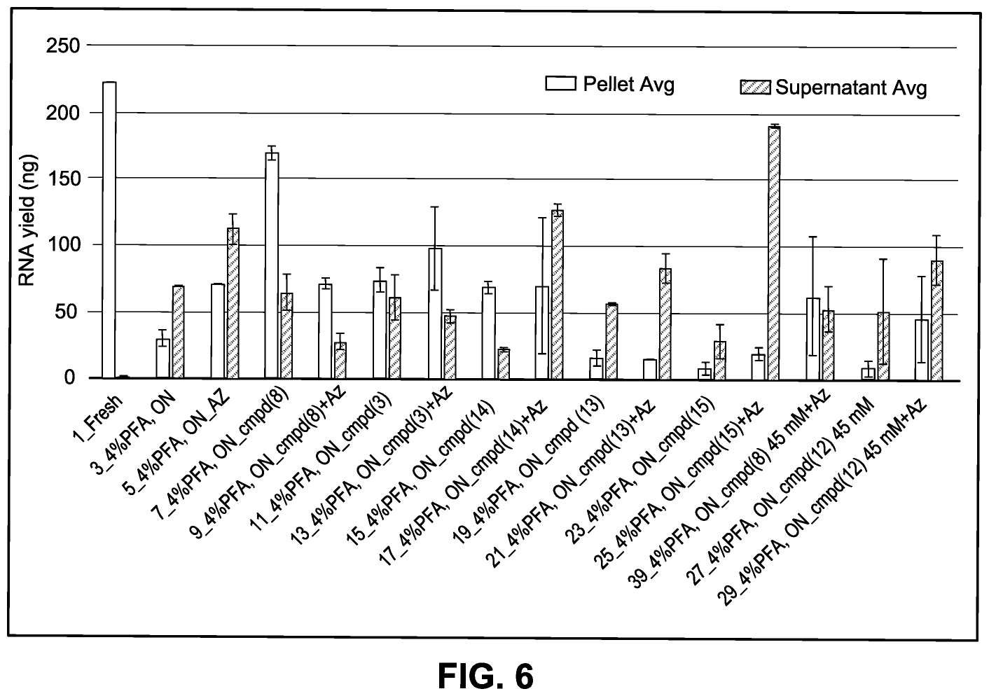

In FIG. 6, de-crosslinking was evaluated by determining the amount of RNA recovered from the cell pellet and the supernatant of Jurkat cells. Jurkat cells were treated with 4% PFA overnight at 4° C. The experimental cells were treated with one of compounds (3), (8), (12), (13), (14), or (15) (100 mM, or as otherwise indicated, 53° for 45 minutes and then 85° C. for 5 minutes), in the presence or absence of ArcticZyme proteinse (10 U/mL). Compared to controls, cells treated with one of compounds (3), (8), (12), (13), (14), or (15) generally have increased RNA recovery from the cell pellet and/or cell supernatant.

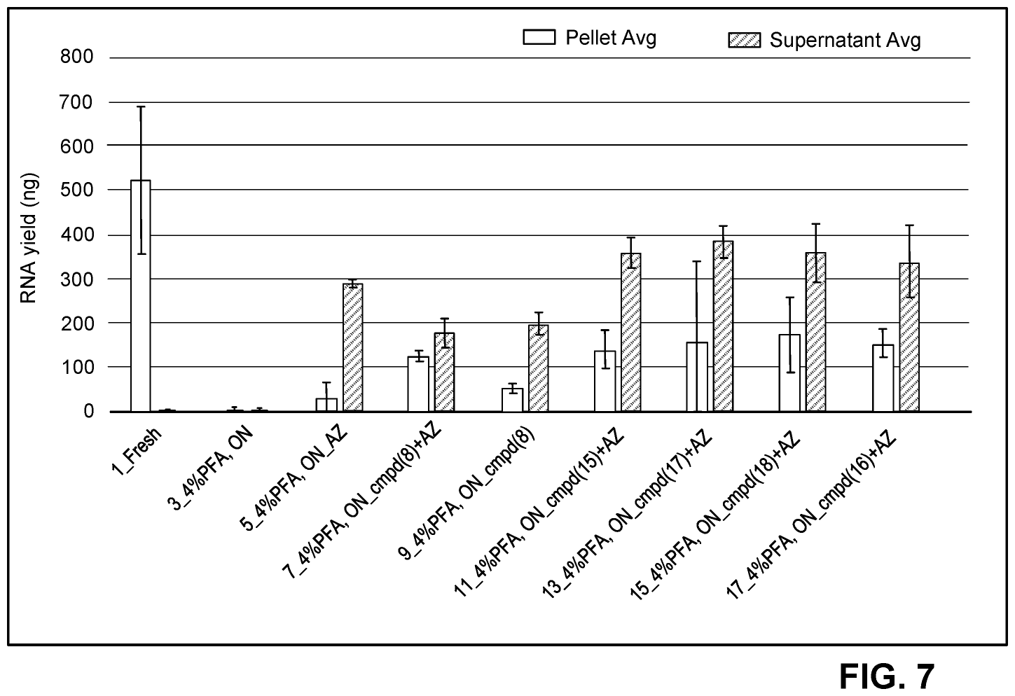

In FIG. 7, de-crosslinking was evaluated by determining the amount of RNA recovered from the cell pellet and the supernatant of Jurkat cells. Jurkat cells were fixed by treating with 4% PFA overnight at 4° C. (about 5 million cells per mL fixative). The experimental cells were treated with one of compounds (8), (15), (16), (17), or (18) (100 mM, or as otherwise indicated), in the presence of ArcticZyme proteinse (10 U/mL) for 90 minutes at 25° C. and then 80° C. for 15 minutes. Compared to controls, cells treated with one of compounds (8), (15), (16), (17), or (18) generally have increased RNA recovery from the cell pellet and/or cell supernatant. From fresh cells, 625 ng of RNA was isolated from the cell pellet.

1-80. (canceled)

81. A method of determining a location of a nucleic acid analyte in a fixed biological sample, the method comprising:
(a) providing an array, wherein the array comprises a plurality of capture probes and wherein a capture probe of the plurality of capture probes comprises: (i) a spatial barcode and (ii) a capture domain;
(b) de-crosslinking the fixed biological sample, wherein at least one of the de-crosslinking agent comprises one or more of:
(i) 2-amino-5-methylbenzoic acid, 2-amino-5-nitrobenzoic acid, 2-amino-5-methylbenzenesulfonic acid, 2,5-diaminobenzenesulfonic acid, 2-amino-3,5-dimethylbenzenesulfonic acid, (2-amino-5-nitrophenyl)phosphonic acid, (4-aminopyridin-3-yl)phosphonic acid, and (2-amino-5-{[2-(2-poly-ethoxy)ethyl]carbamoyl}phenyl)phosphonic acid, or a pharmaceutically acceptable salt thereof; and/or
(ii) the de-crosslinking agent comprises formula (I) 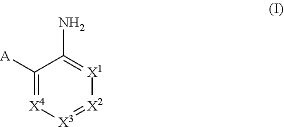
or a pharmaceutically acceptable salt thereof,
wherein A is P(═O)(OH)₂, and wherein each of X¹, X², X³, and X⁴ is CH; and/or
(iii) the de-crosslinking agent comprises formula (I): 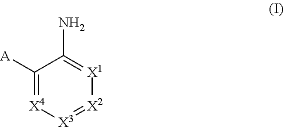
or a pharmaceutically acceptable salt thereof,
wherein:
A is P(═O)(OH)₂ or C(═O)OH;
each of X¹, X², X³, and X⁴ is, independently, CH, CR^{a}, or N;
each R^{a} is, independently, C₁₋₆ alkyl, C₁₋₆ haloalkyl, NO₂, NR′R″, or C(═O)NR′R″;
each of R′ and R″ is, independently, H or optionally substituted C₁₋₆ alkyl; and
at least one of X¹, X², X³, and X⁴ is N; and/or
(iv) the de-crosslinking agent comprises formula (IB): 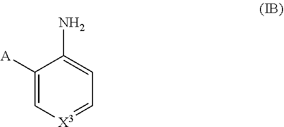
or a pharmaceutically acceptable salt thereof,
wherein A is P(═O)(OH)₂; and
X³ is CH or N;
(c) hybridizing the nucleic acid analyte of the fixed biological sample to the capture domain; and
(d) determining (I) all or a portion of the sequence of the nucleic acid analyte or a complement thereof, and (II) the sequence of the spatial barcode or a complement thereof, and using the determined sequences of (I) and (II) to determine the location of the nucleic acid analyte in the fixed biological sample.

82. The method of claim 81, wherein the fixed biological sample is disposed on the array.

83. The method of claim 81, wherein the fixed biological sample is disposed on a substrate.

84. The method of claim 83, wherein the method further comprises aligning the substrate comprising the fixed biological sample with the array comprising the plurality of capture probes, such that at least a portion of the fixed biological sample is aligned with at least a portion of the array.

85. The method of claim 81, wherein the nucleic acid analyte is RNA.

86. The method of claim 85, wherein the RNA is an mRNA.

87. The method of claim 81, wherein the nucleic acid analyte is DNA.

88. The method of claim 81, wherein the method further comprises extending an end of the capture probe using the nucleic acid analyte as a template, thereby generating an extended capture probe.

89. The method of claim 88, further comprising generating a second strand complementary to the extended capture probe.

90. The method of claim 81, wherein the capture probe further comprises one or more functional domains, a cleavage domain, a unique molecular identifier, and combinations thereof.

91. The method of claim 81, wherein the fixed biological sample is an aldehyde-fixed biological sample or a formaldehyde-fixed biological sample.

92. The method of claim 81, wherein the fixed biological sample is a fixed tissue section.

93. The method of claim 92, wherein the fixed tissue section is a formalin-fixed paraffin-embedded tissue section.

94. The method of claim 93, wherein the formalin-fixed paraffin-embedded (FFPE) tissue section is deparaffinized.

95. The method of claim 94, wherein deparaffinizing the FFPE tissue section comprises, contacting the FFPE tissue section with xylene, absolute ethanol, about 96% ethanol, and about 70% ethanol.

96. The method of claim 95, wherein the FFPE tissue section is:
(i) pre-treated with collagenase at 0.2 U/μL in HBSS buffer for 20 minutes at 37° C.; and/or
(ii) pre-treated with 0.5% of a non-ionic detergent in TE buffer at pH 8 for 20 minutes at 37° C.; and/or
(iii) the FFPE sample is contacted with 50 mM compound (1) in Tris or TE buffer for 1 hour at 70° C.

97. The method of claim 81, wherein the de-crosslinking agent is contacted with the fixed biological sample with one or more of the following conditions:
at a concentration of the de-crosslinking agent from about 10 mM to about 500 mM;
for about 30 minutes to about 90 minutes;
at a pH of about 7.0 to about 9.0; and/or
at a temperature of about 80° C. to about 95° C.

98. The method of claim 81, wherein the de-crosslinking agent comprises Formula IB or a pharmaceutically acceptable salt thereof.

99. The method of claim 81, wherein the de-crosslinking agent is (4-aminopyridin-3-yl)phosphonic acid or a pharmaceutically acceptable salt thereof.

100. A kit for comprising:
(a) a substrate comprising a plurality of capture probes, wherein a capture probe of the plurality of capture probes comprises: (i) a spatial barcode and (ii) a capture domain;
(b) a reagent comprising one or more of compounds (1)-(18);
(c) one or more polymerase enzymes;
(d) one or more wash buffers; and
(e) one or more reaction buffers.
